(19) United States
(12) Patent Application Publication
(10) Pub. No.: US 2005/0036629 A1
(43) Pub. Date: Feb. 17, 2005

(54) Method and device for the interference elimination of a redundant acoustic signal

(76) Inventors: Roland Aubauer, Munchen (DE); Volkmar Hamacher, Neunkirchen am Brand (DE); Stefano Ambrosius Klinke, Kerpen (DE)

Correspondence Address: BELL, BOYD & LLOYD, LLC, P. O. BOX 1135, CHICAGO, IL, 60690-1135, US

(21) Appl. No.: 10/493,283

(22) PCT Filed: Oct. 1, 2002
(86) PCT No.: PCT/EP02/11014

(30) Foreign Application Priority Data
Oct. 22, 2001 (EP) 01125071.9

(51) Int. Cl. A61F 11/06; G10K 11/16; H04B 15/00; H03B 29/00

(52) U.S. Cl. 381/71.1; 381/94.1

(57) ABSTRACT

A method and device are provided for the interference elimination of a redundant acoustic input signal of an acoustic reproducing device, such as a mobile phone or a hearing aid, according to which the interference is concentrated in a partial frequency range of a total frequency range of the input signal. The present invention seeks to improve the sound quality of an acoustic output signal produced. The inventive method includes the following subsequent steps: a) removing the partial frequency range of the input signal, in which the interference is concentrated, b) dividing up the intensity of the frequency range of the input signal that was allowed to pass in the first step into an input signal portion to be maintained and an input signal portion to be further processed, c) synthesizing the partial frequency range of the input signal removed in step a) in accordance with the input signal portion to be further processed, and d) combining the input signal portion from step b) to be maintained and the synthesized input signal portion from step c), thereby producing an output signal that is interference-eliminated as compared to the input signal.

### BACKGROUND OF THE INVENTION

The present invention relates to a method for removing interference from a redundant acoustic input signal to an acoustic reproduction apparatus, such as a mobile telephone or a hearing aid, in which the interference is concentrated in one frequency band element of an overall frequency band of the input signal, and further relates to an apparatus for carrying out such method.

When acoustic signals, such as speech signals, are being received in noisy environments, such as in a motor vehicle, in an industrial environment in which there is machine noise, or in public areas, the problem arises of removing interference from the speech signal before it is reproduced by an acoustic reproduction device. In this case, the aim is to reduce as much as possible undesirable interference signal components in the input signal. A further aim is to prevent artifacts from occurring, including those which affect the interference signal.

It is known to use so-called gradient microphones in order to remove interference from acoustic signals. Such microphones have a directional characteristic which is used to attenuate interference signal components from directions other than the main direction of the microphone, in comparison to a wanted signal in the main microphone direction. However, these microphones have the disadvantage that they are more sensitive to wind and breathing noise. This sensitivity can be improved by the use of wind protection, but this is so large that it cannot be used with all types of acoustic reproduction devices. For example, the use of wind protection is not typically used for a mobile telephone or a hearing aid as the acoustic reproduction devices.

Gradient microphones are normally implemented in hearing aids on the basis of two omnidirectional microphones, using a so-called “delay and subtract” approach. However, the directional effect of this approach, particularly in the lower frequency band of the input signal, is very sensitive to mismatching of the microphone characteristics, which cannot be avoided in practice. Furthermore, there are difficulties with respect to the internal microphone noise.

It is also known for statistical interference noise suppression methods to be used for suppression of interference signals in acoustic signals, such as spectral subtraction methods, the Wiener filter, and methods from Ephraim & Malah, R. Martin, etc. These methods make use of the different statistical characteristics of desired speech signals and of interference signals. It is assumed that an interference signal is considerably more stationary than a desired speech signal. The interference signal component is generally estimated during pauses in speech, and is continuously subtracted on a frequency-specific basis from the input signal that is subject to interference. The estimation of the interference component is particularly difficult in the case of non-stationary interference, particularly when the interference signal is at a higher level than the desired signal.

Artifacts occur during the subtraction of the estimated noise or interference component, and are normally referred to as “musical tones.” Oftentimes, however, the remaining residual interference signal after carrying out the interference noise suppression method no longer has the original interference signal character (that is to say, the noise sounds different), which many users of acoustic reproduction devices are not used to and find disturbing. If the signal-to-noise ratio is low, for example <5 dB, the subtraction principle generally fails, so that the input signal that is subject to interference is generally assessed by the user as sounding better than the processed signal which results after carrying out the interference noise suppression method.

Against this background, the present invention is directed toward a method for removing interference from a redundant acoustic signal in an acoustic reproduction apparatus, and to an apparatus for carrying out such method. Pursuant to such method and apparatus, the sound quality of the resultant acoustic output signal is improved.

### SUMMARY OF THE INVENTION

Accordingly, a method is provided for removing interference from a redundant acoustic input signal to an acoustic reproduction apparatus, such as a mobile telephone or a hearing aid, in which the interference is concentrated in one frequency band element of an overall frequency band of the input signal.

The method includes the following successive steps:
a) removal from the input signal of the frequency band element in which the interference is concentrated;
b) splitting of the intensity of the frequency band of the input signal which is passed through in step a) into an input signal element to be retained and an input signal element to be processed further;
c) synthesis of the frequency band element of the input signal removed in step a) on the basis of the input signal element to be processed further; and
d) combination of the input signal element to be retained from step b) and the synthesized input signal element from step c) in order to produce an output signal with less interference than the input signal.

One major difference between the method of the present invention and the method according to the prior art is that the frequency band element of the input signal in which interference is concentrated can be completely rejected. In contrast, the prior art has always adopted the approach of processing the relevant frequency band element so as to minimize interference.

According to the inventive method, the frequency band element which is removed from the input signal and in which the interference is concentrated is synthesized; in particular, on the basis of a frequency band which has not been removed from the input signal. At this point, use is made of the fact that the input signal element which has not been removed and is intended to be processed further allows conclusions to be drawn about the frequency profile of the frequency band element that has been removed, owing to the redundancy in the acoustic input signal. For example, it can be assumed that an associated frequency spectrum exists for every acoustic sound, in which case a frequency spectrum element can be used to synthesize a spectrum that is adjacent to that frequency spectrum element.

Also, according to the inventive method, the input signal element to be retained, that is to say that part of the overall frequency band of the input signal which is not significantly subject to interference, is combined with the synthesized frequency band element in which the interference does occur, in order to produce an output signal with less interference than the input signal.

The synthesis of the frequency band element of the input signal in step c) is preferably carried out via a bandwidth widening method. For example, in order to widen the bandwidth in the lower frequency band (<300 Hz), it is known from EP 0 994 464 A for signal components in the lower frequency band of a speech signal, whose low frequencies are limited by a high-pass filter, to be reproduced, with the described high-pass filtering being carried out, for example, at the remote subscriber when speech is being transmitted via telephone.

The reproduction is, in this case, carried out by generation of frequencies in the lower frequency band via non-linear signal processing, according to which sub-harmonic frequencies of the signal are produced and are added to the high-pass signal. It should be stressed that the method described in EP 0 994 464 A is not based on the removal of interference from a signal arriving at a mobile telephone or a hearing aid. In contrast, the novel method may be used, inter alia, for mobile telephones, hands-free devices, hearing aids and other communication terminals which are intended for mobile use or use in environments that are subject to interference.

The synthesized frequency band element of the input signal is preferably filtered in step c) for matching to the input signal element of the input signal to be retained. This ensures that the combination process (which is carried out in d)) of these two signal elements leads to an output signal with reduced interference, whose frequency spectrum corresponds as well as possible to the overall frequency spectrum of the wanted signal in the input signal, particularly with regard to the intensity. The characteristics of the spectral filter that is used in step a) also must, of course, be taken into account during the synthesis of that frequency band element of the input signal which is removed or filtered out in step a) because it defines the frequency band which is to be synthesized. Special effects occur for the edge area of a filter that is used.

For many environmental characteristics, such as of a mobile telephone or a hearing aid, it is advantageous for the frequency band element of the input signal which is synthesized in step c) to be at the low-frequency end of the overall frequency band of the input signal. For example, interference in a motor vehicle or so-called pop-and-flow noise caused by wind and breathing typically occurs in the low-frequency region of the input signal. The intensities of the interference frequently are considerably higher than the wanted signal intensities. If attempts are made to overcome this interference using the above-mentioned spectral subtraction method from the prior art, this can be done only by the acceptance of artifacts, whose intensity is high. The method of the present invention is not subject to these restrictions.

Likewise, it is possible to use the inventive method to reduce or prevent interference which is at the high-frequency end of the overall frequency band of the input signal. Even interference which is concentrated in an intermediate frequency band of the overall frequency band of the input signal can be suppressed.

A further advantage of the inventive method is that even highly non-stationary interference can be removed, which is impossible using spectral subtraction methods. The inventive method also can be combined with a gradient microphone, since its disadvantages, such as pop-and-flow noise sensitivity, can be compensated for.

The above-mentioned object is achieved with regard to an apparatus for carrying out the method of the present invention by an apparatus for reproduction of or passing on a redundant acoustic input signal, such as a mobile telephone or a hearing aid, which has interference suppression capabilities for suppression of input signal interference which is concentrated in a frequency band element of the overall frequency band of the input signal. The parts for interference suppression include:
a spectral filter for removal of the frequency band element of the input signal in which the interference is concentrated;
a splitting device, which is arranged downstream from the filter, for splitting the intensity of the frequency band of the input signal that is passed through into an input signal element to be retained and an input signal element to be processed further;
a processing device, which is arranged downstream from the splitting device, and to which the signal element of the input signal to be processed further is supplied, for synthesis of the filtered-out frequency band element of the input signal on the basis of the input signal element to be processed further; and
an output signal production device, to which the input signal element to be retained is supplied from the splitting device, and to which the synthesized input signal element is supplied from the processing device, for combination of the input signal element to be retained and of the synthesized input signal element in order to produce an output signal with less interference than the input signal.

The method of operation of this apparatus is, in fact, evident from the inventive method discussed above, as the apparatus provides the devices that are required for the individual method steps.

It is preferable for the apparatus to have a device for detecting whether the input signal has interference in the cut-off band of the filter, and for a switch to be provided for bridging the interference suppression parts if there is no interference in the cut-off band of the filter. This ensures that the interference suppression parts are switched on only when interference is actually present in the cut-off band of the filter.

It is also advantageous for the apparatus to have a device for detection or estimation of the intensity of any interference, and to have a switch for bridging the interference suppression parts on the basis of the intensity detection/estimation. This makes it possible for the interference suppression parts to be switched on only when this is found to be necessary on the basis of the intensity of the interference. In this case, it is also possible to manually set a threshold value for the detection or estimation of the intensity of any interference; for example, by the user of a mobile telephone. Instead of the switch for bridging the interference suppression parts, a device for overlaying the input signal element in the cut-off band of the filter and of the synthesized signal element, as a function of the amount of interference in the cut-off band of the filter, also may be provided.

With regard to both the method and the apparatus, it should be stressed that any interference may, in principle, be concentrated in any given frequency band element of an overall frequency band of an acoustic signal. The respectively removed or filtered-out frequency band element is then preferably synthesized via a bandwidth widening method, and is combined with the retained frequency band, which was not filtered out, in order to produce an output signal with reduced interference.

Additional features and advantages of the present invention are described in, and will be apparent from, the following Detailed Description of the Invention and the Figures.

### BRIEF DESCRIPTION OF THE FIGURES

FIG. 1 shows a block diagram of an apparatus for removing interference from a redundant acoustic input signal.

### DETAILED DESCRIPTION OF THE INVENTION

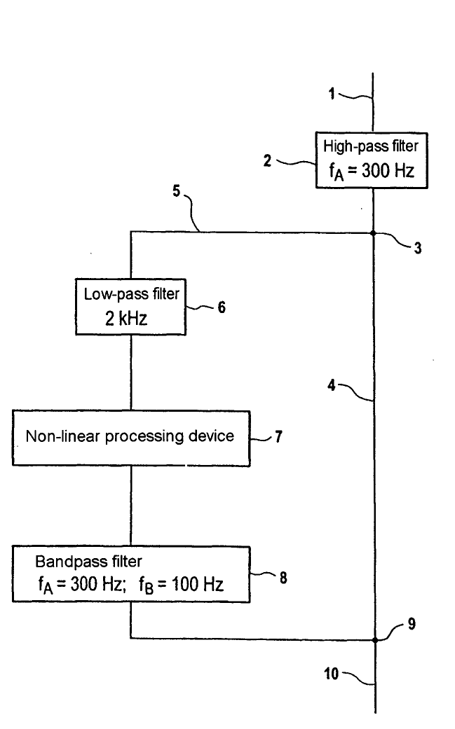

FIG. 1 shows a method procedure and components required for carrying out the method in order to improve the sound of a redundant acoustic input signal 1 that is subject to interference, such as a speech signal. Speech signals have the intrinsic feature that the frequency spectrum that is present at any specific time is not all required to make a statement on what sound should be associated with the current frequency spectrum. To this extent, speech signals are redundant; that is, some of the frequency spectrum can be dispensed with, particularly without any loss of information about the overall frequency spectrum. Such concept is based on the fact that the frequency spectrum element which has been dispensed with can be deduced from the retained frequency spectrum.

In detail and as can be seen in FIG. 1, the input signal 1 (after it has been received by a receiving module, for example, a mobile telephone or hearing aid) is passed to a spectral high-pass filter 2 which has a defined filter characteristic which, in the present exemplary embodiment, allows frequencies above 300 Hz to be passed with virtually no attenuation, while frequencies below 300 Hz are highly attenuated. The value of 300 Hz thus forms the cut-off frequency fA of the high-pass filter 2.

The signal in the frequency band that is passed through is passed from the high-pass filter 2 to a junction point 3 as a splitting device, in which it is split into a signal element 4 to be retained and a signal element 5 to be processed further.

Neither the signal 4 to be retained nor the signal 5 to be processed further has any significant frequency component in the region below 300 Hz, so that any interference which is concentrated below 300 Hz is removed from the input signal 1. To this extent, the explained exemplary embodiment relates to interference which is concentrated at the low-frequency end of the overall frequency band of the input signal 1.

The next method step now includes the synthesis of the frequency spectrum element removed by the high-pass filter 2, to be precise with the aid of the frequency spectrum, which has been passed through, of the signal 5 to be processed further.

The signal 5 to be processed further is first of all passed to a low-pass filter 6, which in the illustrated exemplary embodiment highly attenuates frequencies above 2 kHz. The low-pass filter 6 is not absolutely essential for carrying out the inventive method, but simplifies signal further-processing in a non-linear processing device 7. In particular, the use of the non-linear processing device 7 can result in so-called intermodulation effects occurring, whose appearance is reduced or avoided by the low-pass filter 6.

The non-linear processing device 7 now produces a low-frequency spectrum either directly from the signal 5 to be processed further or from an output signal from the low-pass filter 6; to be precise, taking into account the characteristic of the high-pass filter 2, which is known in advance and is taken into account directly in the non-linear signal processing. The non-linear processing device 7 is used to reproduce amplitudes at the fundamental frequency and/or missing harmonics as a function of the basic speech signal.

The signal which leaves the non-linear processing device 7 thus ideally includes the fundamental frequency and the missing harmonics of the input signal 1, which have been removed in the high-pass filter 2, together with the interference.

The output signal in the form of a synthesized speech signal from the non-linear processing device 7 is then passed to a bandpass filter 8, which passes frequencies below the cut-off frequency fA of 300 Hz of the high-pass filter 2 and a low cut-off frequency fB of, for example, 100 Hz. In principle, the bandpass filter 8 also may be replaced by a low-pass filter with a cut-off frequency of 300 Hz. However, if it is intended to remove DC voltage components from the output signal from the non-linear processing device 7, suitable filtering should be carried out by the bandpass filter 8.

An output signal from the bandpass filter 8 is passed to a linking point 9 as an output signal production device, at which it is added to the signal element 4 to be retained in order to widen the bandwidth, and in order to obtain an output signal 10 with reduced interference, which ideally would have all of the speech wanted signal frequencies of the input signal 1 with an appropriate amplitude.

The apparatus, as illustrated in FIG. 1, for removing interference from the input signal 1 also may be modified such that interference which is at the high-frequency end of the frequency spectrum of the input signal 1 is removed by a low-pass filter, in which case it then would be necessary to provide a non-linear processing unit which can carry out a bandwidth widening process in the direction of high frequencies. In the case of high-frequency interference, the low-pass filter 6 from FIG. 1 would have to be replaced by a suitable high-pass filter. The bandpass filter 8 also would have to be replaced by a filter which allows suitable linking of the synthesized signal element and the signal element 4 to be retained at the linking point 9.

The interference may also occur within the overall frequency band of the input signal 1. In this case, either the frequency band of the input signal 1 at the high-frequency end of the interference or the frequency band of the input signal 1 at the low-frequency end of the interference can be used to carry out a bandwidth widening method, with the non-linear processing unit being used to close a frequency gap that is produced by a bandpass filter instead of the high-pass filter 2.

In further embodiments of the present invention, which are not illustrated in FIG. 1, parts may be provided for switching on the apparatus according to FIG. 1 as a function of external parameters. Examples which may be mentioned include a device for detecting whether the input signal has interference in the cut-off band of the high-pass filter 2, or a device for detection or estimation of the intensity of any interference. In both cases, the respective device is designed to operate a switch which is arranged upstream of the high-pass filter 2.

It is particularly advantageous to provide a device for overlaying the input signal 1 in the cut-off band of the respectively used filter with the synthesized signal element, instead of a hard switch. The degree of overlaying may be dependent on the intensity of any interference that is present. A device such as this for controlling overlaying of the type mentioned may be provided, for example, at the linking point 9, with the device being supplied with an estimated or measured value of the intensity of an interference signal as a control variable.

Although the present invention has been described with reference to specific embodiments, those of skill in the art will recognize that changes may be made thereto without departing from the spirit and scope of the present invention as set forth in the hereafter appended claims.

1-8. (canceled)

9. A method for removing interference from a redundant acoustic input signal to an acoustic reproduction apparatus, wherein the interference is concentrated in one frequency band element of an overall frequency band of the input signal, the method comprising:
a) removing from the input signal the frequency band element in which the interference is concentrated;
b) splitting an intensity of the frequency band of the input signal which is passed through in a) into an input signal element to be retained and an input signal element to be processed further;
c) synthesizing the frequency band element of the input signal removed in a) based on the input signal element to be processed further; and
d) combining the input signal element to be retained from b) and the synthesized input signal element from c) to produce an output signal with less interference than the input signal.

10. A method for removing interference from a redundant acoustic input signal to an acoustic reproduction apparatus as claimed in claim 9, wherein the synthesis of the frequency band element of the input signal in c) is carried out via a bandwidth widening method.

11. A method for removing interference from a redundant acoustic input signal to an acoustic reproduction apparatus as claimed in claim 9, wherein the synthesized frequency band element of the input signal is filtered in c) for matching to the input signal element of the input signal to be retained.

12. A method for removing interference from a redundant acoustic input signal to an acoustic reproduction apparatus as claimed in claim 9, wherein the frequency band element of the input signal synthesized in c) is at a low-frequency end of the overall frequency band of the input signal.

13. A method for removing interference from a redundant acoustic input signal to an acoustic reproduction apparatus as claimed in claim 9, wherein the acoustic reproduction apparatus is one of a mobile telephone and a hearing aid.

14. An apparatus for one of reproducing and passing on a redundant acoustic input signal which includes interference suppression parts for suppressing input signal interference which is concentrated in a frequency band element of an overall frequency band of the input signal, the interference suppression parts of the apparatus comprising:
a spectral filter for removing the frequency band element of the input signal in which the interference is concentrated;
a splitting device, arranged downstream from the filter, for splitting an intensity of the frequency band of the input signal passed through the filter into an input signal element to be retained and an input signal element to be processed further;
a processing device, arranged downstream from the splitting device and to which the signal element of the input signal to be processed further is supplied, for synthesizing the filtered-out frequency band element of the input signal based on the input signal element to be processed further; and
an output signal production device, to which the input signal element to be retained is supplied from the splitting device and to which the synthesized input signal element is supplied from the processing device, for combining the input signal element to be retained and the synthesized input signal element so as to produce an output signal with less interference than the input signal.

15. An apparatus as claimed in claim 14, further comprising:
a device for detecting whether the input signal has interference in a cut-off band of the filter; and
a switch for bridging the interference suppression parts if there is no interference in the cut-off band of the filter.

16. An apparatus as claimed in claim 14, further comprising:
a device for one of detecting and estimating an intensity of any interference; and
a switch for bridging the interference suppression parts based on the intensity of the interference.

17. An apparatus as claimed in claim 14, further comprising:
a device for one of detecting and estimating an intensity of any interference; and
a device for overlaying the input signal element in a cut-off band of the filter and of the synthesized input signal element as a function of the intensity of the interference.

18. An apparatus as claimed in claim 14, wherein the apparatus is one of a mobile telephone and a hearing aid.
